SEQUENCE LISTING (8 sequences)

SEQ ID NO: 1 - DNA, 27 bp, Artificial sequence
  Synthetic oligonucleotide
shaaaaaaaaaaatccttatcaatatt

SEQ ID NO: 2 - DNA, 44 bp, Artificial sequence
  Synthetic oligonucleotide
taggaatagttataagcgtaagtcctaacgaaaaaaaaaachsh

SEQ ID NO: 3 - DNA, 29 bp, Artificial sequence
  Synthetic oligonucleotide
ttataactattcctaaaaaaaaaaachsh

SEQ ID NO: 4 - DNA, 15 bp, Artificial sequence
  Synthetic oligonucleotide
cgttaggacttacgc

SEQ ID NO: 5 - DNA, 29 bp, Artificial sequence
  Synthetic oligonucleotide
taacaataatccctcaaaaaaaaaachsh

SEQ ID NO: 6 - DNA, 15 bp, Artificial sequence
  Synthetic oligonucleotide
gcgtaagtcctaacg

SEQ ID NO: 7 - DNA, 29 bp, Artificial sequence
  Synthetic oligonucleotide
gcgtaagtcctaacgaaaaaaaaaachsh

SEQ ID NO: 8 - DNA, 15 bp, Artificial sequence
  Synthetic oligonucleotide
taggaatagttatta

(19) United States
(12) Patent Application Publication
(10) Pub. No.: US 2009/0280188 A1
(43) Pub. Date: Nov. 12, 2009

(54) ASYMMETRIC FUNCTIONALIZATED NANOPARTICLES AND METHODS OF USE

(75) Inventors: Chad A. Mirkin, Wilmette, IL (US); Fengwei Huo, Evanston, IL (US); Abigail K.R. Lytton-Jean, Chicago, IL (US); Xiaoyang Xu, Evanston, IL (US); Nathaniel L. Rosi, Chicago, IL (US); Yuhuang Wang, Evanston, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: Northwestern University, Evanston, IL (US)

(21) Appl. No.: 12/302,465

(22) PCT Filed: Jun. 25, 2007
(86) PCT No.: PCT/US07/72045

§ 371 Date: Jan. 9, 2009

Related U.S. Application Data
(60) Provisional application No. 60/816,103, filed on Jun. 23, 2006.

(51) Int. Cl. A61K 9/14; A61K 31/7088

(52) U.S. Cl. 424/499; 514/44 A

(57) ABSTRACT

Disclosed herein are asymmetrically functionalized nanoparticles. Further disclosed herein are methods of preparing asymmetrically functionalized nanoparticles. Asymmetrically functionalized nanoparticles can be used in various therapeutic methods.

### CROSS REFERENCE TO RELATED APPLICATIONS

This application claims the benefit of U.S. Provisional Application Ser. No. 60/816,103, filed Jun. 23, 2006, which is incorporated herein in its entirety by reference

### STATEMENT OF GOVERNMENT INTEREST

This invention was made with U.S. government support under Air Force of Scientific Research grant no. FA9550-05-1-0054 and National Institutes of Health/National Cancer Institute grant no. 1U54-CA 119341-01. The government has certain rights in this invention.

### BACKGROUND

The asymmetric functionalization of nanoparticles is a challenging endeavor but one that would open opportunities for synthesizing a new class of materials with properties that derive from the particles themselves and their controlled placement within extended structures (Mirkin, et al., Nature, 382: 607 (1996); Kwon, et al., J. Am. Chem. Soc., 127:10269 (2005); Gu, et al., J. Am. Chem. Soc., 126:5664 (2004); Lu, et al., J. Am. Chem. Soc. 125:12724 (2003); Lu, et al., Nano Lett., 5:379 (2005); Alivisatos, et al., Nature, 382:609 (1996); and Mucic, et al., J. Am. Chem. Soc. 120:12674 (1998)). There are two main challenges in this regard. One pertains to the selective placement of different molecules on different hemispheres or discrete locations on the particle, and the second involves the use of such asymmetry in the generation of assembled architectures not easily attainable with isotropically functionalized materials. DNA functionalized gold nanoparticles are an excellent model system for developing such capabilities. They have been used to identify a variety of new fundamental properties and to develop several useful therapeutic materials and diagnostic systems for nucleic acids, proteins, duplex and triplex DNA binding molecules, and metal ions (Rosi, et al., Science, 312:1027 (2006); Cobbe, et al., J. Phys. Chem. B, 107:470 (1997); Elghanian, et al., Science, 277:1078 (1997); Han, et al., J. Am. Chem. Soc., 128:4954 (2006); He, et al., J. Am. Chem. Soc., 122:9071 (2000); Li, et al, J. Am. Chem. Soc., 126:10958 (2004); Liu, et al., J. Am. Chem. Soc., 125:6642 (2003); Lytton-Jean, et al., J. Am. Chem. Soc., 127:12754 (2005); Nam, et al, Science, 301:1884 (2003); Hazarika, et al, Small, 1:844 (2005); Sato, et al., J. Am. Chem. Soc., 125:8102 (2003); Tato, et al., Science, 289:1757 (2000); Weizmann, et al., The Analyst, 126:1502 (2001); Zhao, et al., J. Am. Chem. Soc., 125:11474 (2003); and Han et al., Angew. Chem. Int. Ed, 45:1807 (2006)). They also have been utilized to demonstrate the concept of programmable materials assembly through the use of their sequence-specific molecular recognition properties. If one could selectively modify a pseudo-spherical nanoparticle (or highly faceted) with different oligonucleotides at specific locations on the particle surface, one could substantially increase the sophistication of the programmable materials synthesis approach (Zanchet et al., Nano Lett., 1:32 (2001)).

Attempts to selectively functionalize very small particles (<10 nm) with long oligonucleotides and subsequently separate them via electrophoretic means have realized some preliminary success (Claridge, et al., Chem. Mater., 17:1628 (2005); Deng, et al., Angew. Chem. Int. Ed., 44:3582 (2005); and Fu, et al., J. Am. Chem. Soc., 126:10832 (2004)). A kinetic control approach has been developed which allows one to functionalize nanoparticles with as few as one oligonucleotide per particle. This strategy introduces anisotropy into such particles and has enabled the assembly of dimer and trimer structures not attainable with the isotropically functionalized particles. Although this was an important step forward in nanoparticle functionalization, it has been limited to very small particles and typically leads to mixtures of products that must be separated by electrophoretic means.

These asymmetrically functionalized particles have been used to synthesize novel nanostructures including dimers, trimers, and one-dimensional arrays. The current limitations of this approach are: 1) the small scale nature of the synthetic procedure, 2) short oligonucleotides (<50 base pairs) cannot be used, and 3) it is limited to small particles, as asymmetrically functionalized particles larger than 10 nm in diameter cannot be efficiently separated via the electrophoretic method. Thus, a need exists for a more efficient and reliable means of providing asymmetrically functionalized nanoparticles, which had the adaptability to be functionalized with a wide range of moieties.

### SUMMARY

Disclosed herein are asymmetrically functionalized nanoparticles. Thus, one aspect of the disclosure provides an asymmetric nanoparticle comprising (1) a first oligonucleotide associated with said nanoparticle, said first oligonucleotide having a first nucleobase sequence comprising about 10 to about 100 nucleobases and (2) a second oligonucleotide associated with said nanoparticle, said second oligonucleotide having a second nucleobase sequence comprising about 10 to about 100 nucleobases, said nanoparticle being greater than 10 nm in diameter, said first nucleobase sequence being different from said second nucleobase sequence, wherein the first oligonucleotide and the second oligonucleotide are concentrated, e.g., anisotropically distributed, at one or more discrete locations on said nanoparticle surface.

In various aspects of the invention, the nanoparticle further comprises a third oligonucleotide associated with said nanoparticle, said third oligonucleotide having a third nucleobase sequence comprising about 10 to about 100 nucleobases, said third nucleobase sequence being different from said first nucleobase sequence and from said second nucleobase sequence. In some embodiments, the third oligonucleotide is concentrated at one or more discrete locations, e.g., anisotropically distributed, on said nanoparticle surface. In various embodiments, the third oligonucleotide is associated with said nanoparticle by hybridization to said first oligonucleotide or to said second oligonucleotide. In some embodiments, said third oligonucleotide is associated with said nanoparticle by covalent interaction.

In various aspects, the nanoparticle further comprises a fourth oligonucleotide having a fourth nucleobase sequence comprising about 10 to about 100 nucleobases, wherein said fourth oligonucleotide is associated with said nanoparticle by hybridization to said first oligonucleotide, said fourth nucleobase sequence sufficiently complementary to said first nucleobase sequence so as to allow hybridization between said fourth oligonucleotide and first oligonucleotide.

In various aspects, said first oligonucleotide or said second oligonucleotide is associated with said nanoparticle by covalent interaction. Alternatively or additionally, said first oligonucleotide and said second oligonucleotide are associated with said nanoparticle by covalent interaction.

Also provided herein are complexes comprising a first nanoparticle and a second nanoparticle as disclosed herein, said first nanoparticle having a diameter of about 10 to about 25 nm, and said second nanoparticle having a diameter of about 30 to about 60 nm, wherein said first nucleobase sequence associated with said first nanoparticle is sufficiently complementary to said first nucleobase sequence associated with said second nanoparticle to permit hybridization therewith, and wherein said first oligonucleotide associated with said first nanoparticle and said first oligonucleotide associated with said second nanoparticle are hybridized.

In various aspects, the complex comprises a third nanoparticle, said third nanoparticle having a diameter of about 65 to about 100 nm, said first nucleobase sequence associated with said third nanoparticle being sufficiently complementary to said second nucleobase sequence associated with said second nanoparticle to permit hybridization therewith, wherein said first oligonucleotide associated with said third nanoparticle and said second oligonucleotide associated with said second nanoparticle are hybridized.

Also provided herein are methods of preparing an asymmetric nanoparticle as disclosed herein comprising the step of adding a ligase to an admixture comprising (a) a microparticle having a surface functionalized with a first oligonucleotide having a first nucleobase sequence comprising about 10 to about 50 nucleobases, (b) a second oligonucleotide having a second nucleobase sequence comprising about 10 to about 100 nucleobases and either a 3′ hydroxyl functional group or a 5′ phosphate functional group, said second nucleobase sequence being sufficiently complementary to a first region of said first nucleobase sequence to allow said second oligonucleotide to hybridize to said first oligonucleotide, and (c) a gold nanoparticle having a surface functionalized with a third oligonucleotide having a third nucleobase sequence comprising about 10 to about 100 nucleobases and either a 5′-phosphate functional group or a 3′ hydroxyl functional group, said third nucleobase sequence being sufficiently complementary to a second region of said first oligonucleotide, wherein, when said second oligonucleotide and said third oligonucleotide are hybridized to said first oligonucleotide, said first region and said second region are adjacent such that said functional group of said second oligonucleotide and said functional group of said third oligonucleotide are positioned to permit ligation between said second oligonucleotide and said third oligonucleotide; under conditions appropriate to ligate said second oligonucleotide and said third oligonucleotide to provide said asymmetric gold nanoparticle. In various aspects of the methods, the nanoparticle has a diameter of about 10 to about 100 nm. In some aspects, the microparticle has a diameter of at least about 150 nm. In specific embodiments, the microparticle is magnetic.

In various aspects, the method further comprises separating said microparticle associated with said asymmetric nanoparticle from the admixture and releasing the asymmetric nanoparticle from the microparticle. In embodiments where the microparticle is magnetic, the separation can be via magenetic separation. In some embodiments, the separating comprises chromatography or sedimentation. In specific embodiments, the chromatography comprises use of size exclusion chromatography or affinity chromatography.

In certain aspects, the releasing is via heating the mixture to melt said double stranded complex.

Also provided are methods of preparing an asymmetric nanoparticle comprising the steps of a) admixing, under conditions to permit hybridization, (1) a microparticle having a double stranded complex comprising a first oligonucleotide and a second oligonucleotide, and (2) a first gold nanoparticle having a diameter of about 10 nm to about 100 nm and comprising a third oligonucleotide associated with said nanoparticle, said first oligonucleotide having a first nucleobase sequence comprising about 10 to about 50 nucleobases, said second oligonucleotide being associated with the surface of said microparticle via covalent interaction and having a second nucleobase sequence comprising about 10 to about 50 nucleobases, said second nucleobase sequence having about 5 to about 10 contiguous nucleobases that are sufficiently complementary to a first end of the first nucleobase sequence to form said double stranded complex on said microparticle, said third oligonucleotide having a third nucleobase sequence comprising about 15 to about 50 nucleobases in which a sequence of more than 10 contiguous nucleobases in said third nucleobase sequence is sufficiently complementary to a second end of said first nucleobase sequence, such that said first and said third oligonucleotide are hybridized to from a second double stranded complex; and b) subjecting the admixture of step (a) to a temperature sufficient to melt said first double stranded complex and insufficient to melt said second double stranded complex, to produce said asymmetric gold nanoparticle.

Further disclosed are methods of delivering a therapeutic into a cell comprising contacting a cell with an asymmetric nanoparticle as disclosed herein, wherein said first oligonucleotide is bound to the therapeutic.

In various aspects, the second oligonucleotide is bound to an agent that facilitates entry of the nanoparticle into the cell.

In various aspects, the therapeutic is a protein, a peptide-modified nucleic acid, a neutral-modified nucleic acid, drug molecule, gene, or siRNA.

### BRIEF DESCRIPTION OF THE DRAWINGS

FIG. 1(A) shows a synthetic scheme for the asymmetric functionalization of nanoparticles with DNA, and FIG. 1(B) shows satellite nanostructures how are formed by hybridizing asymmetrically functionalized 13 nm gold nanoparticles with symmetrically functionalized 20 nm gold nanoparticles.

FIG. 2 shows a transmission electron microscope (TEM) image of satellite structures formed using the disclosed methods, e.g., SiO₂ particles modified with gold nanoparticles, such that only a few oligonucleotides on one hemisphere of each of the nanoparticles hybridize to the central SiO₂ particle.

FIG. 3 shows TEM images of satellite structures composed of 13 nm asymmetrically functionalized gold nanoparticles and 20 nm symmetrically functionalized gold nanoparticles.

FIG. 4 shows UV-Vis spectra of satellite nanostructures (top trace) and unhybridized mixture of 13 and 20 nm gold nanoparticles (bottom trace).

FIG. 5(A) shows Dynamic Light Scattering (DLS) data for satellite nanostructures, and FIG. 5(B) shows a schematic depicting the diameter of the satellite nanostructures.

FIG. 6 shows a schematic of a method of preparing asymmetric nanoparticles as disclosed herein using magnetic microparticles and ligating oligonucleotides on a nanoparticle's surface in an asymmetric fashion.

FIG. 7A shows DNA melting curves of 13 nm gold nanoparticles (AuNPs) that are hybridized with magnetic microparticles (MMPs) through an “extension” DNA. The ligation step significantly increased the melting temperature, from 54° C., before (upper curve), to 74.5° C., after (lower curve). Without the “extension” DNA, the ligation step showed minimal effect (middle curve).

FIG. 7B shows scanning electron microscope (SEM) image of 30 nm AuNPs on the surface of a MMP.

FIGS. 8A and 8B depict schematic (8A) and TEM (8B) images of asymmetric nanoparticles in a “cat paw” macrostructure; FIGS. 8C and 8D depict the “satellite” structure; and FIGS. 8E and 8F depict the dendrimer-like structure.

FIG. 9 shows the UV-Vis spectra of 13-20 nm AuNPs satellite structures (top (first) spectrum), the dispersed 13 nm AuNPs (second spectrum), the dispersed 30 nm AuNPs (third spectrum), and a mixture of the 13 nm and 30 nm AuNPs without hybridization.

FIG. 10 shows the dynamic light scattering (DLS) measurements of 13 nm AuNPs functionalized with DNA (left most spectrum −36±6 nm), 30 nm AuNPs functionalized with DNA (center spectrum −58±3 nm), and the satellite structures (right most spectrum −152±10 nm). The bottom figure shows a model of the satellite structures with the estimated sizes of the various components.

FIG. 11 shows a TEM image of three-component AuNP dendrimer-like structures.

FIG. 12 shows confocal fluorescence microscopy images showing use of oligonucleotide-modified gold nanoparticles for EGFP knockdown in cells. 12A. Untreated control cells. 12B. 1 μm sectioning images of control cells in 12A. 12C. Cells treated with antisense particles showed a decrease in the amount of EGFP emission. 12D. 1 μm sectioning images of cells in 12C.

FIG. 13 shows a schematic for asymmetric functionalization of gold nanoparticles with directionally added components for cellular delivery. One face of the particle (oligonucleotide rich) is used for transfection, while the other is used to carry the protein cargo.

### DETAILED DESCRIPTION

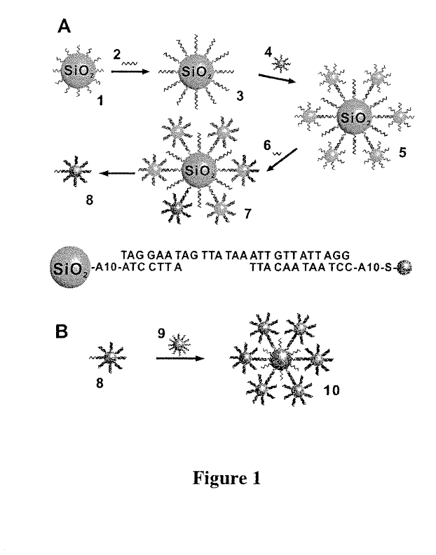

Methods are disclosed herein for synthesizing nanoparticles asymmetrically functionalized with oligonucleotides that provide excellent control over the placement of oligonucleotides on one hemisphere of the nanoparticle surface. These particles can be prepared on a relatively large scale, and the synthetic procedure is independent of particle size (FIG. 1A). Also disclosed are methods of using the asymmetric nanoparticles, including, but not limited to, as therapeutics, as delivery vehicles, and/or both.

Gold nanoparticles (AuNPs) can be anisotropically functionalized with two or more different oligonucleotide sequences using magnetic microparticles as geometric restriction templates for site-selective enzymatic extension of particle-bound oligonucleotides. The divalent linking capability of the resulting AuNPs allowed for the design and programmable assembly of discrete nanoparticle heterostructures.

Programmable assembly methods based upon the use of oligonucleotide-functionalized nanoparticles and sequence-specific assembly with complementary DNA have led to the development of a variety of fundamentally interesting materials and technologically significant detection systems. One feature of this approach to materials synthesis is that one can control the size, shape, and compositions of the individual nanoparticle building blocks as well as their spacing and periodicity within a macroscopic and often times polymeric structure through judicious choice of nanoparticle building block and DNA linkers. Most of the work in this area has focused on the use of isotropically functionalized particles since there are very few ways of selectively functionalizing different surface regions of an individual particle. However, if one could deliberately functionalize only one hemisphere or one distinct point on a particle in a general way, one could begin to introduce valency into such structures, thereby allowing greater control over the assembly process.

### Asymmetric Nanoparticles

The term “asymmetric nanoparticle” as used herein refers to a nanoparticle having a surfaced modified with more than one oligonucleotide/oligonucleotide sequence, wherein the nanoparticles have at least one oligonucleotide sequence concentrated at least one discrete location of the nanoparticle while the other oligonucleotide sequence(s) is/are less concentrated at that same location. These locations can be interspersed throughout the surface of the nanoparticle, located on one half of the surface of the asymmetric gold nanoparticle, or be at only one location on the surface. As used herein, “concentrated” means that a particular location on the surface of the asymmetric gold nanoparticle, one oligonucleotide species is greater than 50%, greater than about 55%, greater than about 60%, greater than about 65%, greater than about 70%, greater than about 75%, greater than about 80%, greater than about 85%, greater than about 90%, greater than about 95%, greater than about 96%, greater than about 97%, greater than about 98%, and greater than about 99% of all oligonucleotides at that location. Nanoparticles modified in this way differ from those previously known in the art which have different oligonucleotides randomly located on the nanoparticle surface. The nanoparticle can be comprised of any material that can be associated with oligonucleotides and preferably does not interfere, inhibit, or otherwise distort the desired oligonucleotide activity or properties. Other materials contemplated include those that are disclosed in WO 06/138145.

In various aspects, the nanoparticles disclosed herein have a size of about 10 nm to about 250 nm in mean diameter, about 10 nm to about 240 nm in mean diameter, about 10 nm to about 230 nm in mean diameter, about 10 nm to about 220 nm in mean diameter, about 10 nm to about 210 nm in mean diameter, about 10 nm to about 200 nm in mean diameter, about 10 nm to about 190 nm in mean diameter, about 10 nm to about 180 nm in mean diameter, about 10 nm to about 170 nm in mean diameter, about 10 nm to about 160 nm in mean diameter, about 10 nm to about 150 nm in mean diameter, about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter, about 10 nm to about 10 nm in mean diameter. In other aspects, the size of the nanoparticles is from about 15 nm to about 150 nm (mean diameter), from about 15 to about 50 nm, from about 10 to about 30 nm. The size of the nanoparticles is from about 15 nm to about 150 nm (mean diameter), from about 30 to about 100 nm, from about 40 to about 80 nm. Also contemplated are nanoparticles of about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 205 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, and about 300 nm.

The size of the nanoparticles used in a method varies as required by their particular use or application. The variation of size is advantageously used to optimize certain physical characteristics of the nanoparticles, for example, optical properties or amount surface area that can be derivatized as described herein or in preparing the asymmetrically functionalized nanoparticles. For example, in preparing the asymmetrically functionalized nanoparticles as disclosed herein, two or more nanoparticles are employed, each having a different size than the other. The size differences are used to allow for the blocking of one side of the smaller nanoparticle and permit asymmetric functionalization of that smaller nanoparticle. Thus, while the asymmetrically functionalized nanoparticles can be of a size as disclosed above, the relative size of the nanoparticles is selected in either used in the methods of preparing the asymmetrically functionalized nanoparticles or in the structures of two or more asymmetrically functionalized nanoparticles (e.g., catpaw, dendrimer, or satellite structures) to allow for the formation of the structures or asymmetrically functionalized nanoparticles of interest.

The terms “associated with” or “attached to,” as used herein, refer to an interaction between the surface of the nanoparticle and the oligonucleotide. That interaction can be through any means. Regardless of the means by which the oligonucleotide is attached to or associated with the nanoparticle, attachment in various aspects is effected through a 5′ linkage, a 3′ linkage, some type of internal linkage, or any combination of these attachments. In some embodiments, the association is via a covalent interaction. Other means of association are also contemplated, such as ionic interaction, van der Waals interactions, hydrophobic interactions, and mixtures of such interactions.

The term “oligonucleotides” as used herein includes modified forms as discussed herein as well as those otherwise known in the art. Likewise, the term “nucleotides” as used herein is interchangeable with modified forms as discussed herein and otherwise known in the art. In certain instances, the art uses the term “nucleobase” which embraces naturally-occurring nucleotides as well as modifications of nucleotides that can be polymerized into an oligonucleotide and has specific hybridization characteristics. Nonlimiting examples include compounds such as heterocyclic compounds that can serve like nucleobases including certain “universal bases” that are not nucleosidic bases in the most classical sense but serve as nucleosidic bases. Especially mentioned as universal bases are 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Nanoparticles for use in the methods provided are functionalized with an oligonucleotide, or modified form thereof, which is from about 5 to about 100 nucleotides in length. Methods are also contemplated wherein the oligonucleotide is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated. Also contemplated are oligonucleotides of about 10 to about 250 nucleobases.

The use of ordinals (e.g., “first” or “second” or “third” and so forth) to refer to elements such as an nanoparticles, oligonucleotides, and nucleobase sequences is for clarity purposes only, to identify which nanoparticles, oligonucleotides, and nucleobase sequences are related to each other and to distinguish the oligonucleotides and nucleobase sequences of one nanoparticle from the oligonucleotides and nucleobase sequences of another nanoparticle. The ordinals are not meant to imply any particular relationship or required order between the multiple nanoparticles, oligonucleotides, and/or nucleobase sequences.

Oligonucleotides may also include base modifications or substitutions. As used herein, “unmodified” or “natural” nucleobases include the purine bases adenine (A) and guanine (G), and the pyrimidine bases thymine (T), cytosine (C) and uracil (U). Modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. In certain aspects, the modified base provides a Tₘ differential of 15, 12, 10, 8, 6, 4, or 2° C. or less. Exemplary modified bases are described in EP 1 072 679 and WO 97/12896. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Ang Chem Int E, 30: 613 (1991), and those disclosed by Sanghvi, Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference. Non-naturally occurring nucleobases are also contemplated, such as, but not limited to, xanthine, diaminopurine, 8-oxo-N-6-methyladenine, 7-deazaxanthine, 7-deazaguanine, N4,N4-ethanocytosin, N′,N′-ethano-2,6-diaminopu-rine, 5-methylcytosine (mC), 5-(C₃-C₆)-alkynyl-cytosine, 5-fluorouracil, 5-bromouracil, pseudoisocytosine, 2-hydroxy-5-methyl-4-triazolopyridin, isocytosine, isoguanine, inosine. Also contemplated are “non-naturally occurring” nucleobases described in Benner et al., U.S. Pat. No. 5,432,272 and Susan M. Freier and Karl-Heinz Altmann, Nuc Acid Res, 25:4429-4443 (1997). The term “nucleobase” thus includes not only the known purine and pyrimidine heterocycles, but also heterocyclic analogues and tautomers thereof. Further naturally and non-naturally occurring nucleobases include those disclosed in U.S. Pat. No. 3,687,808, in Chapter 15 by Sanghvi, in Antisense Research and Application, eds. S. T. Crooke and B. Lebleu, CRC Press, 1993, in Englisch et al., Ang. Chem., Int Ed, 1991, 30, 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety).

“Hybridization” means an interaction between two strands of nucleic acids by hydrogen bonds in accordance with the rules of Watson-Crick DNA complementarity, Hoogstein binding, or other sequence-specific binding known in the art. Hybridization can be performed under different stringency conditions known in the art.

In various aspects, the nanoparticles and methods disclosed herein include use of an oligonucleotide which is 100% complementary to the target oligonucleotide, i.e., a perfect match, while in other aspects, the oligonucleotide is at least (meaning greater than or equal to) about 95% complementary to the target oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the target oligonucleotide to the extent that the oligonucleotide is able to hybridize to the target oligonucleotide under a particular stringency condition. The complementarity can be concentrated at a specific region of the oligonucleotide or can be along the entire length of the oligonucleotide.

“Stringency conditions” for hybridization is a term of art which refers to the incubation and wash conditions, e.g., conditions of temperature and buffer concentration, which permit hybridization of a particular oligonucleotide to a second oligonucleotide; the first oligonucleotide may be perfectly (i.e., 100%) complementary to the second, or the first and second may share some degree of complementarity that is less than perfect (e.g., 70%, 75%, 85%, 95%). For example, certain high stringency conditions can be used which distinguish perfectly complementary oligonucleotides from those of less complementarity. “High stringency conditions”, “moderate stringency conditions” and “low stringency conditions” for oligonucleotide hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the oligonucleotide sequence, nucleobase composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two oligonucleotides. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology 200: 546-556 (1991), and in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of about 17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a prewarmed solution (42° C.) solution containing 0.2×SSC/0.1% SDS for 15 minutes at 42° C.; and a high stringency wash can comprise washing in prewarmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The term “melts” is understood in the art to mean dissociation of hybridized polynucleotides, generally brought about by an increase in temperature to greater than a “melting temperature, Tₘ.” Changes in environmental conditions can alter the Tₘ for any given hybridization complex, such conditions including for example, pH, salt concentration, and the concentration of other hybridization mixture additives known in the art.

The term “double stranded complex” is used herein to refer to the hybridized complex of two oligonucleotides.

The asymmetric nanoparticles can be prepared using a larger microparticle of the same or different material, wherein the nanoparticle and microparticle associate via oligonucleotides on each of their surfaces and oligonucleotide(s) not associate with a micro- or nanoparticle. A ligase is added to a mixture comprising
(a) a microparticle having a surface functionalized with a first oligonucleotide having a first nucleobase sequence comprising about 10 to about 50 nucleobases,
(b) a second oligonucleotide having a second nucleobase sequence comprising about 10 to about 50 nucleobases and either a 3′ hydroxyl functional group or a 5′ phosphate functional group, said second nucleobase sequence being sufficiently complementary to a first region of said first nucleobase sequence to allow said second oligonucleotide to hybridize to said first oligonucleotide, and
(c) a gold nanoparticle having a surface functionalized with a third oligonucleotide having a third nucleobase sequence comprising about 10 to about 50 nucleobases and either a 5′-phosphate functional group or a 3′ hydroxyl functional group, said third nucleobase sequence being sufficiently complementary to a second region of said first oligonucleotide, wherein, when said second oligonucleotide and said third oligonucleotide are hybridized to said first oligonucleotide, said first region and said second region are adjacent such that said functional group of said second oligonucleotide and said functional group of said third oligonucleotide are positioned to permit ligation between said second oligonucleotide and said third oligonucleotide;
   under conditions appropriate to ligate said second oligonucleotide and said third oligonucleotide to provide said asymmetric gold nanoparticle.

In some embodiments, the method further comprises separating the ligated mixture and releasing the asymmetric nanoparticle from the microparticle. The separating can be via any known means of the art. In embodiments where the microparticle is magnetic, the separating can be via application of a magnetic field. In embodiments where the microparticle is associate with a surface (e.g., glass slide), the separating can be via removal of the surface from the admixture (e.g., removing a glass slide having an associated microparticle from a solution having the gold nanoparticle, ligase, and second oligonucleotide). In various embodiments, the separating can be via chromatography, e.g., size exclusion or affinity chromatography. In embodiments where the separating is via affinity chromatography, the microparticle can be modified to further include an appropriate affinity tag. For example, the microparticle can comprise a protein or oligonucleotide for protein-antibody or oligonucleotide-antibody affinity chromatography, streptavidin or biotin, or a histidine tag for metal-protein affinity chromatography (e.g., histidine—nickel affinity chromatography). In some embodiments, the separating can be via sedimentation, wherein complexes of different masses or densities are separated. The means of modifying a particle with a biomolecule, such as a protein, histidine tag, and/or streptavidin or biotin are known in the art, and are described, for example, in U.S. Pat. Nos. 5,635,602; 5,665,539; 6,495,324; 6,506,564; 6,582,921; 6,602,669; 6,610,491; 6,645,721; 6,673,548; 6,677,122; 6,682,895; 6,709,825; 6,720,147; 6,720,411; 6,726,847; 6,730,269; 6,740,491; 6,750,016; 6759199; 6767702; 6773884; and 6777186; and International Publication Nos. WO/2001/073123 and WO/2001/051665.

A therapeutic, as used herein, is any compound, structure, or biomolecule which exhibits therapeutic properties. Such entities include, but are not limited to, small molecule drugs, proteins, peptides, organometallic therapeutics (e.g., cis-platnin), siRNA, and the like. Also contemplated are therapeutics of modified nucleic acids, such as peptide-modified nucleic acids or nucleic acids that are neutrally modified. Oligonucleotide-drug conjugates and their preparation are described in U.S. Pat. No. 6,656,730, which is incorporated herein by reference in its entirety. See, for example U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, the disclosures of which are incorporated herein by reference.

The term “siRNA” describes a technique by which post-transcriptional gene silencing (PTGS) is induced by the direct introduction of double stranded RNA (dsRNA: a mixture of both sense and antisense strands). (Fire et al., Nature 391:806-811, 1998). Current models of PTGS indicate that short stretches of interfering dsRNAs (21-23 nucleotides; siRNA also known as “guide RNAs”) mediate PTGS. The siRNAs are apparently produced by cleavage of dsRNA introduced directly or via a transgene or virus. The siRNA modified nanoparticles, therefore, are oligonucleotides that have a 21-23 nucleobase component that is transferred into the cell with the nanoparticle in order to promote or facilitate RNAi. Preparation and use of RNAi compounds is described in U.S. Patent Application No. 2004/0023390, the disclosure of which is incorporated herein by reference in its entirety.

### Methods of Using Asymmetrically Functionalized Nanoparticles

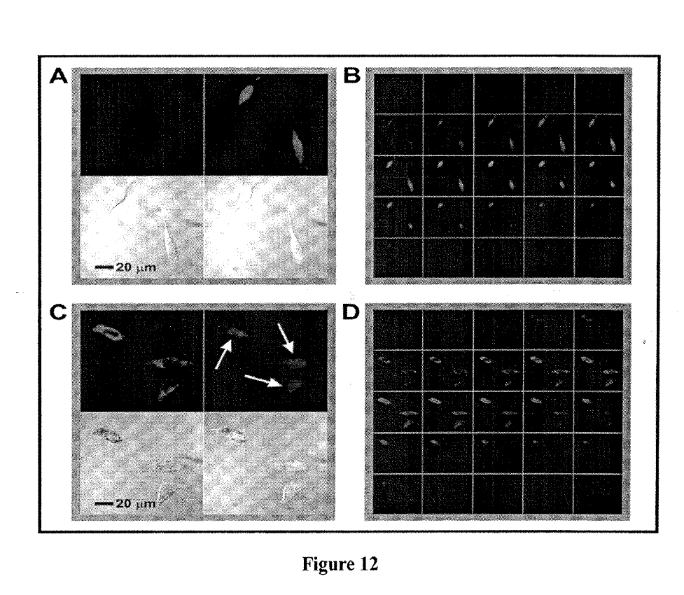

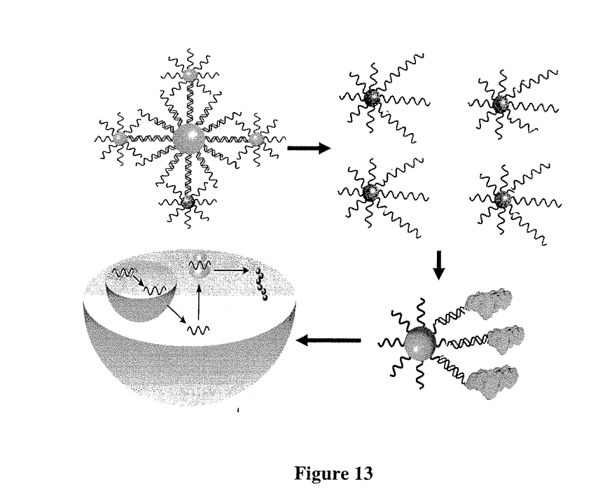

The control of intercellular protein expression with oligonucleotide functionalized gold nanoparticles (DNA-NPs) has been recently disclosed in International Publication No. WO 06/138145. Gold nanoparticles have proven to be an effective carrier that enable the introduction of oligonucleotides into a diverse sampling of cell types without the use of traditional transfection agents. Thus, these DNA-NPs are a potentially powerful new way of regulating cellular gene expression (FIG. 12). Importantly, the ability to systematically control the oligonucleotide loading on the nanoparticle surface make them not only attractive candidates for antisense studies and therapies, but for use as carrier agents as well. Attaching the oligonucleotides to the gold nanoparticle surface creates cooperative properties including high binding affinity, high serum stability and efficiency of cellular entry, when compared to the analogous free oligonucleotides. Additionally, the use of the gold nanoparticle has shown no observable toxicity and allows for determination of entry characteristics through the visualization of the NP with microscopy techniques. These oligonucleotide-modified gold nanoparticles will perform as very effective multifunctional carrier vehicles for delivery across cellular membranes.

One of the primary challenges in delivery of molecules into cells is passing the cellular membrane. Oligonucleotide-modified gold nanoparticles are able to enter all cell types that have thus far been tested. This is surprising, as most transfection agents rely on positively charged carriers such as lipoplexes or polyplexes in order to achieve DNA entry into cells. Thus, the mechanism of entry of the oligonucleotide-NPs is of particular interest. Without being bound by theory, it is possible that positively charged proteins within the cell culture environment are binding to the negatively charged DNA backbone. Upon binding, the particle/protein complex may become positively charged and effect entry into the cells.

The entry properties of nanoparticles themselves can be exploited for use of the oligonucleotide-modified particles as carriers of biological and chemical materials across the cell membrane. The DNA-NP conjugate seems to be unique in its ability to enter cells without the use of additional transfection agents and displays much more rapid and effective than either component alone. For example, particles that are functionalized with other surface moieties have not shown comparable ability to enter cells (Tkachenko, et al., J Am Chem Soc, 125:4700-4701 (2003)). Thus, the densely packed DNA that is presented to the cell plays a role in the uptake kinetics of the system.

In other systems, including lipoplexes and polyplexes that have been used to carry molecules into cells, toxicity has been an issue. Toxicity does not seem to be a limiting factor with the gold nanoparticle system. In fact, viability has been so remarkable that a “toxic limit” on the amount of oligonucleotide-modified nanoparticles that can be added has yet been reached. In fact, even at high (0.12 nmol) loadings, evaluation of the toxicity in multiple cell lines has not shown any appreciable cell death.

Another significant challenge for introducing molecules into cells is determining the percentage of uptake, which is usually dependent on co-transfection of a secondary reporter. The nanoparticles overcome this limitation, as evaluation of metal content using inductively coupled plasma mass spectrometry (ICP-MS) permits determination of the number of gold nanoparticles that accumulate inside the cellular environment over time. By measuring the starting concentration of particles added, determination of the number of particles inside the cells after specific time points is possible. This rate has proven to be cell type dependent and also modifiable by varying the concentrations of nanoparticles that are presented to the cells. This determination is useful for equalizing concentrations of cargo molecules that are added, as the properties of the nanoparticle allow for tracking as well as simple determination of particle entry and thus payload delivery.

The oligonucleotide-nanoparticle conjugate should retain characteristic entry properties while systematically adding functionality. Asymmetrically functionalized nanoparticles with two different oligonucleotides in a site-dependent manner can allow for this bifunctional requirement of entry properties and therapeutic functionality.

Linker sequences and aptamers to complementary molecules of interest including proteins, plasmids, antibodies, peptides, and the like can be designed into the asymmetrically functionalized nanoparticles, which can then can be spatially restricted around the nanoparticle. The properties of the densely packed DNA oligonucleotides on one area of the particle will be effective in causing entry across cellular membranes, while this additional functionality will be able to specifically hybridize and carry specific molecules of interest into the cell (FIG. 13). Once inside the cell, this cargo may be released either by specifically designed enzymatic degradation, hydrolysis, ligand exchange or near IR heating of the cells.

The ability to not only deliver proteins or other therapeutics across cell membranes in a non-toxic manner, but also to determine the efficiencies of entry and delivery of the particular load without the use of a secondary reporter is an extremely valuable and versatile tool for facilitating studies in cellular models. By using asymmetrically designed gold nanoparticle complexes as platforms for delivery, a new class of multifunctional therapeutics are disclosed that take advantage of the cooperative properties of the oligonucleotides on the nanoparticle surface for rapid and efficient cellular entry.

### EXAMPLES

### Formation of Asymmetric Nanoparticles

### Example 1

Citrate-stabilized 13 nm gold nanoparticles were synthesized according to literature methods (Lytton-Jean, et al., J. Am. Chem. Soc., 127:12754 (2005)). 20 nm gold nanoparticles were purchased from Polyscience, Inc. Thiol-modified and amine-modified oligonucleotides were synthesized and coupled to gold and silica surfaces, respectively, by previously described methods (Rosi, et al., Angew Chem Int Ed, 43:5500 (2004)). The sequence that was attached to the gold nanoparticles was a 3′-thiol modified sequence (TTA CAA TAA TCC-A₁₀-5H-3′ (SEQ ID NO:2)). The sequence that was attached to the silica particles was SEQ ID NO. 1. The size and optical properties of the particles did not significantly change upon modification with the oligonucleotides as examined by TEM and UV-Vis spectroscopy. The concentration of 13 nm gold nanoparticles and 20 nm gold nanoparticles were determined by UV-Vis spectroscopy (extinction coefficient: 2.7×108 M⁻¹ cm⁻¹ at λ₅₂₀ for 13 nm gold nanoparticles and 1.2×109 M⁻¹ cm⁻¹ at λ₅₂₆ for 20 nm gold nanoparticles). Asymmetrically functionalized 13 nm gold nanoparticles are mixed with 20 nm gold nanoparticles functionalized with complementary oligonucleotides (molar ratio 10:1) in 0.4 M NaCl, 10 mM PBS, 0.1% SDS. The sample was left to shake overnight, allowing DNA hybridization to occur and then centrifuged to remove excess 13 nm gold nanoparticles.

### Example 2

Oligonucleotide linkers are used to connect oligonucleotide-modified gold nanoparticles to a larger oligonucleotide-modified SiO₂ particle to form a satellite structure 5 (FIG. 1A). The resulting oligonucleotide duplexes that interconnect the satellite structure are thermally addressable at two different sites, one adjacent to the SiO₂ particle (7-mer) and the other near the gold particle (12-mer). Since these two duplexes are different lengths, they melt at different temperatures (Tₘs), allowing one to release the gold nanoparticles with the linker intact yielding an exposed “sticky end” 8.

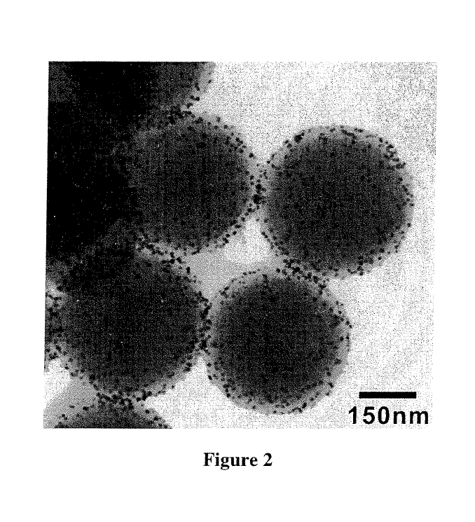

7-mer oligonucleotide-modified SiO₂ particles (Rosi, et al., Angew Chem Int Ed, 45:5500 (2004) 1 were hybridized with a 27-mer oligonucleotide containing a 7-mer complementary region 2, which results in a particle with many duplexes with 20-mer overhanging ends 3. These particles were then hybridized to gold nanoparticles functionalized with 12-mer oligonucleotides 4 that are complementary to the overhanging portion of 3. The 12-mer and 7-mer duplexes melt at 35° C. and 23° C., respectively, in 0.10 M NaCl, 10 mM PBS buffer, allowing one too independently and sequentially address the structures with temperature. When a 1,000:1 molar ratio of gold nanoparticles to SiO₂ is used, satellite structures 5 are formed such that only a few oligonucleotides on one hemisphere of each of the nanoparticles hybridize to the central SiO₂ particle (FIG. 2). The remaining oligonucleotides on the gold nanoparticle surface are then blocked by forming duplexes with 12-mer oligonucleotides 6. Since the Tₘs of 7-mer and 12-mer duplexes that connect the gold nanoparticle and SiO₂ particle differ by 12° C., one can selectively dehybridize the 7-mer regions by increasing the temperature above the Tₘ for the 7-mer duplexes while remaining below the Tₘ for the 12-mer structures. This liberates asymmetrically functionalized gold nanoparticles 8 which posses overhanging oligonucleotides with “sticky ends” only at the points of contact between the gold nanoparticles and the larger SiO₂ particles.

By using these asymmetrically functionalized particles, structures that are not easily accessible through the use of symmetrically functionalized particles can be obtained. To demonstrate this capability, the asymmetrically functionalized 13 nm particles 8 described above are combined with 20 nm gold particles 9 functionalized with complementary DNA (5′SH-A10-ATC CTT ATC AAT ATT 3′ (SEQ ID NO: 1)) at a 10:1 ratio, FIG. 1B. Because the 13 nm particles are asymmetrically functionalized, satellite structures 10 form as opposed to the polymeric aggregates that typically form with isotropically functionalized particles at a 1:1 to 10:1 ratio. With the asymmetrically functionalized particles, a 10:1 ratio leads to complete formation of satellite structures with a small amount of unbound 13 nm particles. Larger ratios (>15:1) lead to larger amounts of 13 nm particles remaining in solution, but large aggregates are not observed. Smaller ratios (<10:1) lead to incomplete satellite structure formation. Note that analogues of these satellite structures have been made in low yield using large ratios of complementary isotropically functionalized particles.

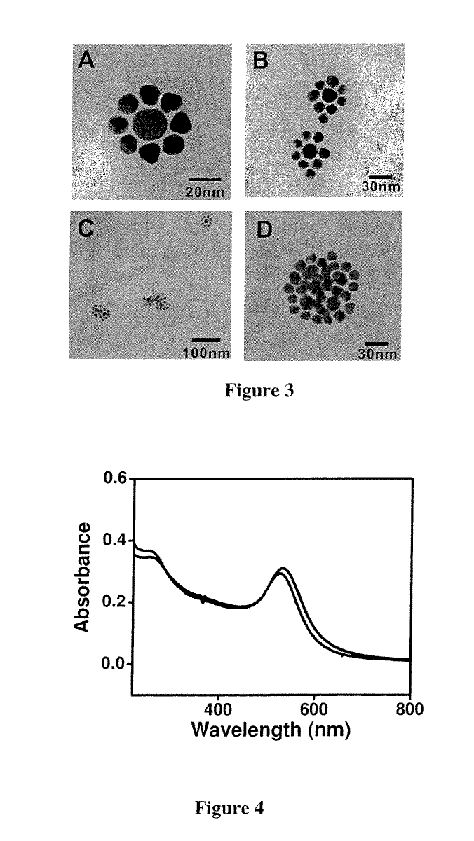

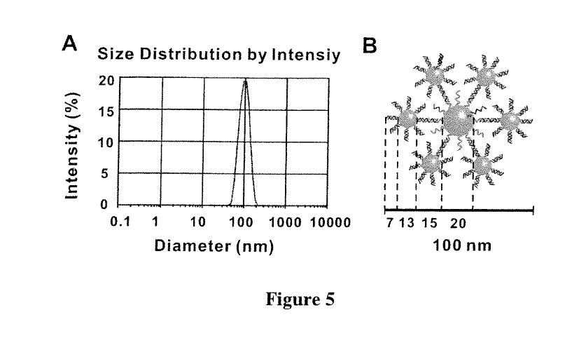

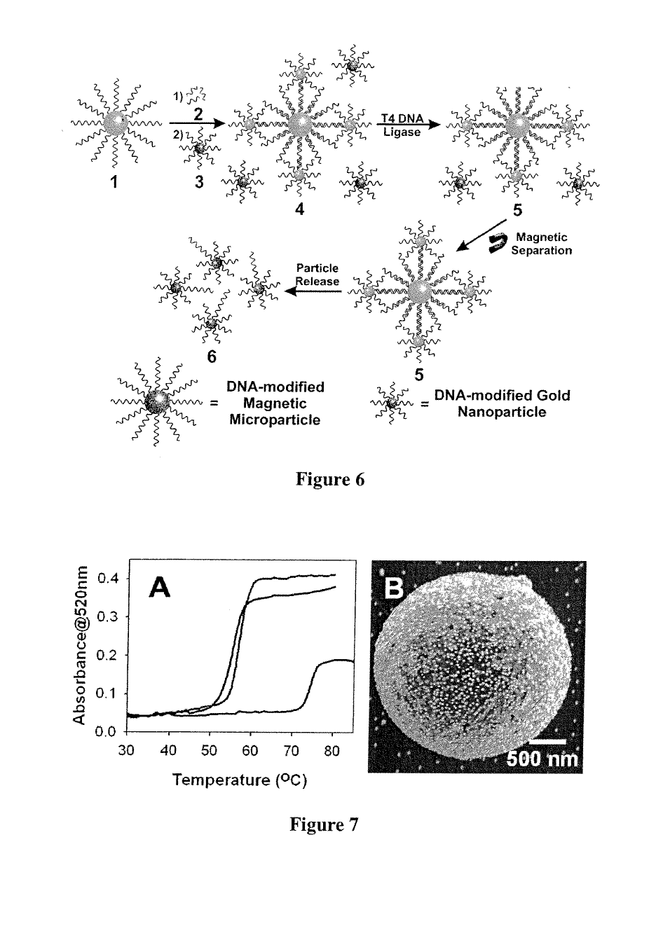

TEM, UV-Vis spectroscopy, and light scattering measurements were carried out to characterize the assembled nanostructures. Satellite structures, composed of 20 nm gold particles surrounded by several 13 nm gold particles, were characterized by TEM (FIG. 3). However, in these cases, TEM is not the ideal technique for characterization as drying effects can lead to sample clumping which is not representative of the solution phase (FIGS. 3C and 3D). More appropriate characterization techniques include UV-Vis spectroscopy and Dynamic Light Scattering (DLS). In the UV-Vis absorbance spectrum, a peak at 533 nm supports the formation of satellite structures. This is a 6 nm red shift from the 527 nm band characteristic of the unhybridized mixture of 13 and 20 nm gold particles, FIG. 4. The small red shift is associated with the formation of small aggregates; larger aggregates would result in a much larger (e.g., about 60 nm) red shift. DLS measurements also support this conclusion showing a single band consistent with the formation of about 100 nm diameter structures, approximately the diameter of the proposed satellite structure, FIG. 5.

Functionalized Magnetic MicroParticles (MMPS) with DNA

Magnetic microparticles were functionalized with DNA using a known methods (Stoeva, et al., Angew. Chem. Int. Ed., 45:3303-3306 (2006)). Commercially available aminofunctionalized magnetic microparticles (MMPs, Dynal Biotech, Dynabeads M-270 Amine) were activated with a NHS-ester linker, and then coupled with thiol-terminated “template” DNA. Amine-functionalized MMPs (1 mL; 30 mg/mL) were placed on a magnetic stand, collected, washed (3×) with anhydrous DMSO (Aldrich), and then resuspended in succinimidyl 4-(p-maleimidophenyl)butyrate (Pierce)/DMSO solution (15 ml; 10 mM). The suspension was incubated (4 h) with gentle shaking (New Brunswick Scientific, Incubator Shaker, 12400) to activate the amino group. After incubation, the particles were washed (3×) with anhydrous DMSO (10 mL) and then with a coupling buffer (2×) (0.1 M sodium phosphate buffer, pH 7.0 with 0.2 M NaCl). The MMPs were then resuspended in the coupling buffer, and template DNA (SEQ ID NO: 2-5′TAGGAATAGTTATAAGCGTAAGTCCTAACG-A₁₀-(CH₂)₃—SH 3′) was added (0.5 ml; 5 μM). The suspension was sealed with foil and parafilm and then shaken (Eppendorf, Thermomixer R) (1,400 RPM) overnight at room temperature to ensure efficient coupling. After reaction, the supernatant was removed, and the MMPs were washed (3×) with coupling buffer (10 mL) and then with a passivation buffer (2×) (0.15 M sodium phosphate buffer pH 8.0 with 0.15 M NaCl). They were then suspended in Sulfo-NHS acetate (Pierce) (35 ml; 10 mM) and incubated and shaken (1 h) at room temperature. After passivation, the particles were centrifuged (Eppendorf Centrifuge 5415D) (4,000 RPM; 1 min) and washed (3×) with passivation buffer (2×) and then with a storage buffer (2×) (10 mM sodium phosphate buffer pH 7.4 with 0.20 M NaCl). Finally, the particles were resuspended in storage buffer (3 ml) so that their final concentration was 10 mg/ml.

### DNA Ligation on AuNPs

The 13 nm AuNPs were synthesized and functionalized with oligonucleotides according to previously reported methods (Storhoff, et al. J. Am. Chem. Soc., 120:1959-1964 (1998)). The 5′ phosphate DNA was synthesized following literature procedures (Guzaev, et al. Tetrahedron, 51:9375-9384 (1995). The AuNPs were heavily functionalized with 5′ phosphate DNA (SEQ ID NO: 3-5′PO₄³⁻-TTATAACTATTCCTA-A₁₀-(CH₂)₃—SH 3′], which was complementary to half of the template DNA on the MMPs (SEQ ID NO: 2). AuNPs and MMPs were then mixed for hybridization in the presence of the “extension” DNA (SEQ ID NO: 4-^{5′}CGTTAGGACTTACGCOH 3′) and ligation buffer (0.05 M Tris-HCl buffer pH=7.5, 5 mM MgCl₂, 1 mmol ATP, 0.05 mg/ml BSA, T4 DNA Ligase 4,000 units/ml). A thermomixer (Eppendorf) was used to keep the MMPs suspended in solution. After mixing overnight, the MMPs along with hybridized AuNPs were extracted from the reaction mixture by applying a magnetic field. The extracted particles were washed (3×) with PBS (10 mM sodium phosphate buffer pH 7.4 with 0.10 M NaCl) to remove residual oligonucleotides. Because of the diameter of MMPs (2.8 μm) and the surface roughness, the 13 nm AuNPs are too small to hybridize with more than one MMP in solution. The oligonucleotide-functionalized AuNPs will hybridize with MMPs only at their contact points, leaving the oligonucleotides on the other regions of the AuNP unchanged. After hybridization, “template” oligonucleotides on the MMPs co-align the “extension” oligonucleotides and the AuNP oligonucleotides, allowing their enzymatic ligation in the presence of T4 DNA ligase.

Analogous methods were used to asymmetrically functionalize larger particles but with different oligonucleotide sequences. For the 30 nm AuNPs in the “cat paw” structure, the particle DNA is SEQ ID NO: 5 (5′PO₄³⁻-TAACAATAATCCCTC-A₁₀-(CH₂)₃—SH 3′) and the “extension”

DNA is SEQ ID NO: 6 (5′GCGTAAGTCCTAACG-OH3′). For the dendrimer-like structures, the particle DNA for the 60 nm AuNPs is SEQ ID NO: 7 (5′ GCGTAAGTCCTAACG-A₁₀-(CH₂)₃-SH3′); for the 30 nm AuNPs, the particle DNA is SEQ ID NO: 3 and the “extension” DNA is SEQ ID NO: 4; for the 13 nm AuNPs, the particle DNA is SEQ ID NO: 5 and the “extension” DNA is SEQ ID NO: 8 (5′ TAG GAA TAG TTA TTA-OH 3′)

### Self-Assembly of DNA-Asymmetrically Functionalized AuNPs

To obtain the satellite structure, asymmetrically-functionalized 13 nm AuNPs (0.5 ml; 9 nM) were mixed with oligonucleotide-functionalized (SEQ ID NO: 7) 30 nm AuNPs (100 μl; 3 nM) in PBS buffer (10 mM sodium phosphate buffer, pH 7.4 with 0.10 M NaCl) and shaken overnight at room temperature. The satellite structures were collected and purified by repeated centrifugation (Eppendorf Centrifuge 5415D) (6,000 RPM; 6 min) (3×) and disposal of the excess 13 nm AuNPs. Analogous hybridization procedures were used to obtain the “cat paw” and the dendrimer-like structures.

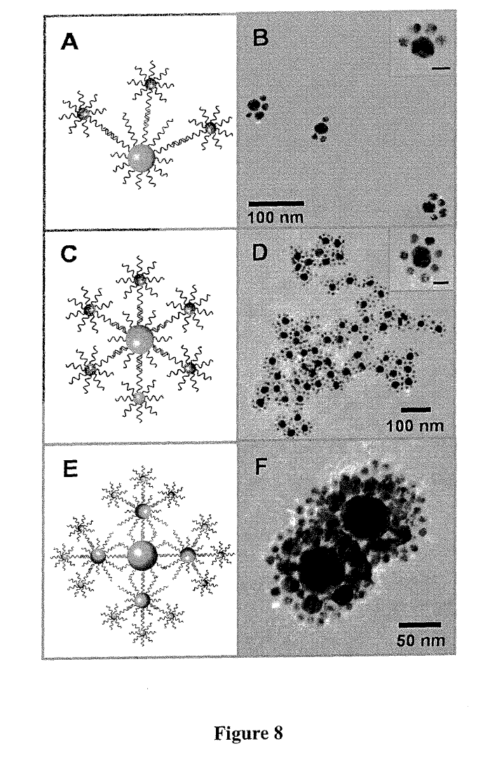

Cat Paw Structures: AuNPs (13 and 30 nm), both of which were asymmetrically-functionalized with complementary extension oligonucleotides, are mixed together. Because only the “extension” oligonucleotides of the AuNPs can hybridize, a “cat paw” structure can be formed (FIGS. 8A and 8B). The “cat paw” structures suggest that for each 30 nm AuNP, approximately ⅓ to ½ of its surface is asymmetrically functionalized with the “extension” oligonucleotides.

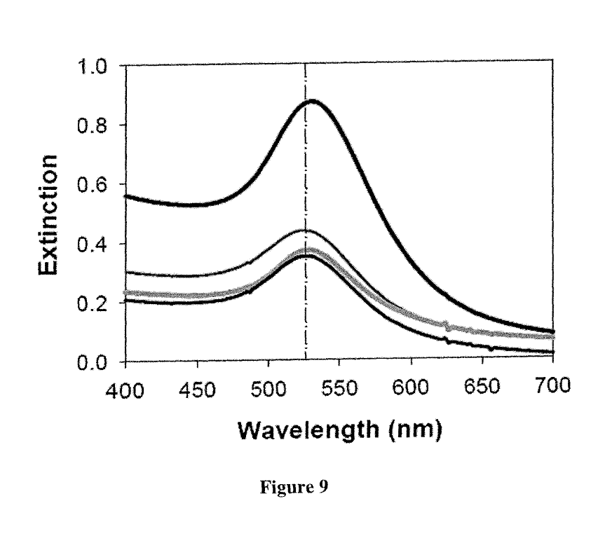

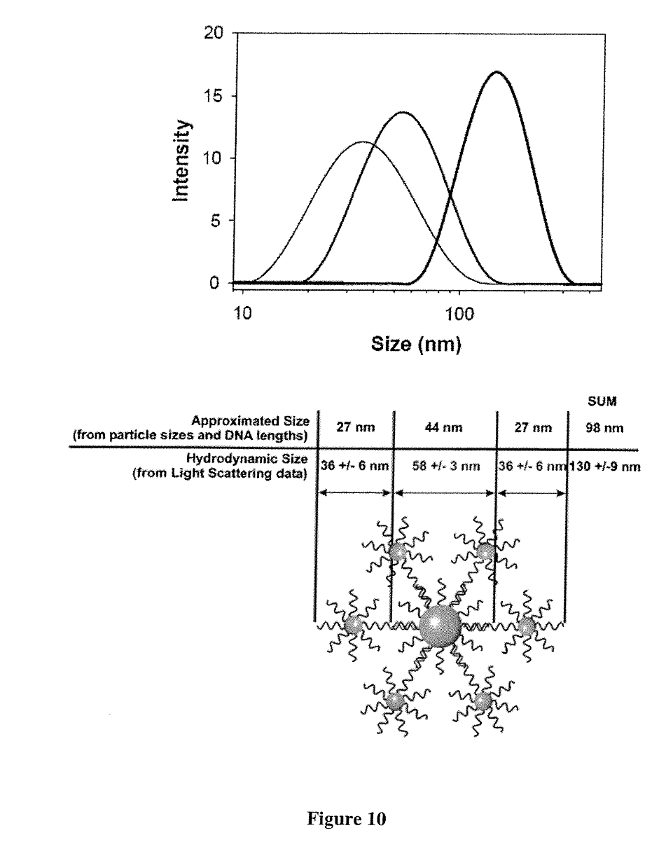

Satellite Structures: Asymmetrically functionalized 13 nm AuNPs can be mixed with 30 nm AuNPs that are functionalized with oligonucleotides complementary only to the extended strands on the 13 nm AuNP. Because of the asymmetric functionalization of AuNP, the two sets of nanoparticles do not aggregate, but instead formed satellite structures consisting of one 30-nm AuNP surrounded by 13 nm AuNPs. TEM analysis of the sample reveals that nearly every 30 nm AuNP was hybridized with six to ten 13 nm AuNPs (FIGS. 8C and 8D). Although drying of the sample and the electron beam can substantially affect the state of the DNA-duplex interconnects, the TEM data does conclusively show that the asymmetry of the AuNPs stops the sequence specific oligomerization process in the form of the satellite structure. Notably, the satellite-like nanoparticle complexes exhibit a 6 nm red shift in their surface plasmon absorption compared with what is normally observed for dispersed 30 nm AuNPs (FIG. 9). This red shift is consistent with Mie theory and the formation of a small aggregate as opposed to the large polymeric structures typically attained with the isotropically functionalized particles. Dynamic light scattering measurements also confirm the formation of satellite structures with an average diameter of 152±10 nm, which is approximately the diameter one would expect from modeling the satellite structure made from the two different sizes of AuNP building blocks and DNA interconnects (FIG. 10).

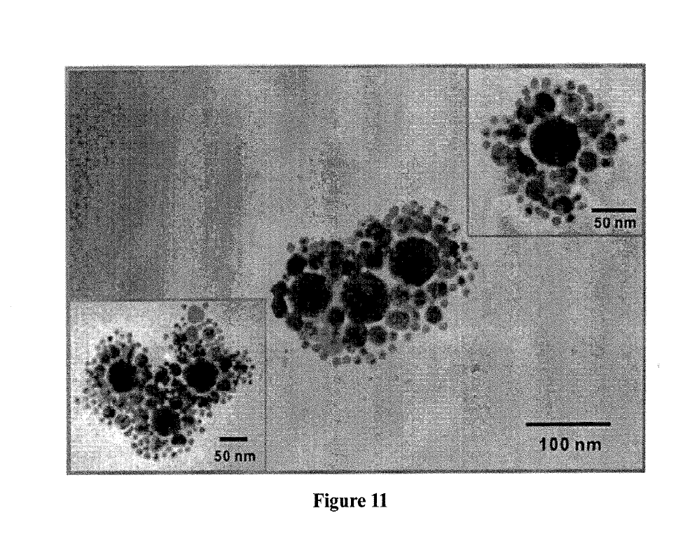

Dendrimer Structures: A third type of nanostructure that resembles a dendrimer is possible through these asymmetrically-functionalized particles. The dendrimer-like structures are formed by further hybridizing the non-extended oligonucleotides on the 30 nm particles of satellite structures with complementary extension oligonucleotides on asymmetrically-functionalized 13 nm AuNPs (FIGS. 8E and 8F and FIG. 11). This three-component structure demonstrates that this method and asymmetrically functionalized particles can be used to precisely control the assembly of at least three different AuNPs into designed heterostructures in a step-by-step fashion.

All of the above U.S. patents, U.S. patent application publications, U.S. patent applications, foreign patents, foreign patent applications and non patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

1. An asymmetric gold nanoparticle comprising (1) a first oligonucleotide associated with said nanoparticle, said first oligonucleotide having a first nucleobase sequence comprising about 10 to about 100 nucleobases and (2) a second oligonucleotide associated with said nanoparticle, said second oligonucleotide having a second nucleobase sequence comprising about 10 to about 100 nucleobases, said nanoparticle being greater than 10 nm in diameter, said first nucleobase sequence being different from said second nucleobase sequence, wherein said first oligonucleotide and said second oligonucleotide are concentrated at one or more discrete locations on said nanoparticle surface.

2. The asymmetric gold nanoparticle of claim 1, further comprising a third oligonucleotide associated with said nanoparticle, said third oligonucleotide having a third nucleobase sequence comprising about 10 to about 100 nucleobases, said third nucleobase sequence being different from said first nucleobase sequence and from said second nucleobase sequence.

3. The asymmetric gold nanoparticle of claim 2, wherein said third oligonucleotide is concentrated at a discrete location on said nanoparticle surface.

4. The asymmetric gold nanoparticle of claim 2, wherein said third oligonucleotide is associated to said nanoparticle by hybridization to said first oligonucleotide or to said second oligonucleotide.

5. The asymmetric gold nanoparticle of claim 2, wherein said third oligonucleotide is associated with said nanoparticle by covalent interaction.

6. The asymmetric gold nanoparticle of claim 1, further comprising a fourth oligonucleotide having a fourth nucleobase sequence comprising about 10 to about 100 nucleobases, wherein said fourth oligonucleotide is associated with said nanoparticle by hybridization to said first oligonucleotide, said fourth nucleobase sequence sufficiently complementary to said first nucleobase sequence so as to allow hybridization between said fourth oligonucleotide and said first oligonucleotide.

7. The nanoparticle of claim 1, wherein said first oligonucleotide or said second oligonucleotide is associated with said nanoparticle by covalent interaction.

8. A complex comprising a first nanoparticle according to claim 1 and a second nanoparticle according to claim 1, said first nanoparticle having a diameter of about 10 to about 25 nm, and said second nanoparticle having a diameter of about 30 to about 60 nm, wherein said first nucleobase sequence associated with said first nanoparticle is sufficiently complementary to said first nucleobase sequence associated with said second nanoparticle to permit hybridization therewith, and wherein said first oligonucleotide associated with said first nanoparticle and said first oligonucleotide associated with said second nanoparticle are hybridized.

9. The complex of claim 8 further comprising a third nanoparticle according to claim 1, said third nanoparticle having a diameter of about 65 to about 100 nm, said first nucleobase sequence associated with said third nanoparticle being sufficiently complementary to said second nucleobase sequence associated with said second nanoparticle to permit hybridization therewith, wherein said first oligonucleotide associated with said third nanoparticle and said second oligonucleotide associated with said second nanoparticle are hybridized.

10. A method of preparing an asymmetric gold nanoparticle comprising
adding a ligase to an admixture comprising
(a) a microparticle having a surface functionalized with a first oligonucleotide having a first nucleobase sequence comprising about 10 to about 50 nucleobases,
(b) a second oligonucleotide having a second nucleobase sequence comprising about 10 to about 50 nucleobases and either a 3′ hydroxyl functional group or a 5′ phosphate functional group, said second nucleobase sequence being sufficiently complementary to a first region of said first nucleobase sequence to allow said second oligonucleotide to hybridize to said first oligonucleotide, and
(c) a gold nanoparticle having a surface functionalized with a third oligonucleotide having a third nucleobase sequence comprising about 10 to about 50 nucleobases and either a 5′-phosphate functional group or a 3′ hydroxyl functional group, said third nucleobase sequence being sufficiently complementary to a second region of said first oligonucleotide, wherein, when said second oligonucleotide and said third oligonucleotide are hybridized to said first oligonucleotide, said first region and said second region are adjacent such that said functional group of said second oligonucleotide and said functional group of said third oligonucleotide are positioned to permit ligation between said second oligonucleotide and said third oligonucleotide; under conditions appropriate to ligate said second oligonucleotide and said third oligonucleotide to provide said asymmetric gold nanoparticle.

11. The method of claim 10, wherein said gold nanoparticle has a diameter of about 10 to about 100 nm.

12. The method of claim 10, wherein said microparticle has a diameter of at least about 150 nm.

13. The method of claim 10, further comprising separating said microparticle associated with said asymmetric nanoparticle from the admixture and releasing said asymmetric nanoparticle from said microparticle.

14. The method of claim 13, wherein said microparticle is magnetic and said separating comprises magnetic separation.

15. The method of claim 13, wherein said separating comprises use of chromatography or sedimentation.

16. The method of claim 15, wherein said separating comprises use of size exclusion chromatography.

17. The method of claim 15, wherein said separating comprises use of affinity chromatography.

18. The method of claim 13, wherein releasing is via heating the mixture to melt said double stranded complex.

19. A method of preparing an asymmetric gold nanoparticle comprising:
a) admixing, under conditions to permit hybridization, (1) a microparticle having a double stranded complex comprising a first oligonucleotide and a second oligonucleotide, and (2) a first gold nanoparticle having a diameter of about 10 nm to about 100 nm and comprising a third oligonucleotide associated with said nanoparticle,
said first oligonucleotide having a first nucleobase sequence comprising about 10 to about 50 nucleobases,
said second oligonucleotide being associated with the surface of said microparticle via covalent interaction and having a second nucleobase sequence comprising about 10 to about 50 nucleobases,
said second nucleobase sequence having about 5 to about 10 contiguous nucleobases that are sufficiently complementary to a first end of the first nucleobase sequence to form said double stranded complex on said microparticle,
said third oligonucleotide having a third nucleobase sequence comprising about 15 to about 50 nucleobases in which a sequence of more than 10 contiguous nucleobases in said third nucleobase sequence is sufficiently complementary to a second end of said first nucleobase sequence, such that said first and said third oligonucleotide are hybridized to from a second double stranded complex; and
b) subjecting the admixture of step (a) to a temperature sufficient to melt said first double stranded complex and insufficient to melt said second double stranded complex, to produce said asymmetric gold nanoparticle.

20. A method of delivering a therapeutic into a cell comprising contacting a cell with an asymmetric gold nanoparticle of claim 1, wherein said first oligonucleotide is bound to the therapeutic.

21. The method of claim 20, wherein the therapeutic is a protein.

22. The method of claim 20, wherein the therapeutic is a peptide-nucleic acid.

23. The method of claim 20, wherein the therapeutic is a drug molecule.

24. The method of claim 20, wherein the therapeutic is a gene.

25. The method of claim 20, wherein the therapeutic is siRNA.
